(19) United States
(12) Patent Application Publication
(10) Pub. No.: US 2013/0184534 A1
(43) Pub. Date: Jul. 18, 2013

(54) SYSTEMS, DEVICES AND METHODS FOR CREATING AN OPERATIVE SITE WITHIN A PATIENT

(71) Applicants: Robert Evans, Chicago, IL (US); Douglas P. Allen, Los Angeles, CA (US)

(72) Inventors: Robert Evans, Chicago, IL (US); Douglas P. Allen, Los Angeles, CA (US)

(21) Appl. No.: 13/348,807

(22) Filed: Jan. 12, 2012

(51) Int. Cl. A61B 1/32

(52) U.S. Cl. 600/207

(57) ABSTRACT

A device for creating an operative space within a body lumen for the performance of a medical procedure, including a pressurizable, expandable balloon element insertable into the body lumen. The balloon element includes an interior space for receiving an inflation fluid. The device further includes an ingress valve coupled with the balloon element and insertable therewith into the body lumen. The ingress valve is capable of being opened to allow the interior space to be filled with the inflation fluid and closed to retain the inflation fluid in the interior space. The device may also include an egress valve coupled with the balloon element and insertable therewith into the body lumen. The egress valve is closed when the interior space is filled with the inflation fluid and is capable of being opened to allow the inflation fluid to vent from the interior space.

### TECHNICAL FIELD

This invention generally relates to systems, devices and methods for creating operative sites and thereby improving visualization during diagnostic and surgical procedures, such as gastrointestinal (GI) procedures. More particularly, the invention relates, but is not limited to, procedures such as Transanal Endoscopic Microsurgery (TEM), where current distension methods can engender complications.

### BACKGROUND

Lower GI procedures typically require the use of a distension media, often carbon dioxide, to create a viable operative site. The use of a gas as a distension media is known as insufflation. Current TEM procedures utilize a sealed transanal cannula for introducing surgical instruments into the GI tract. The cannula includes a sealed cap to maintain insufflation pressure within the GI tract, and also includes several ports. One port is dedicated to a camera and another two or three are dedicated to instruments. Each instrument port is comprised of an inner leaflet valve and an outer seal. These ports restrict the lateral movement of the instrument. They also cause friction drag on the instruments as they are moved axially within the port, thus reducing the tactile feedback that the surgeon experiences. Due to the valve and seal combination, the proximal and distal movements of the instrument within the port are cumbersome.

Adding to the cumbersome nature of the current TEM methods, the pressurized system requires that either the outer seal or the entire cannula sealing cap be removed to allow excised tissue to be extracted from the GI tract. Additionally, a surgeon will sometimes remove the outer seal to introduce a needle into the operative space. It is possible to hold the needle in a grasper such that the needle can be pushed through the seal, but this risks damage to the seal. The repeated removal and replacement of the outer seals or the entire sealing cap is also time consuming and tedious.

At first, room air was used as the distension gas as it was plentiful and efficient to use. However, it was discovered that CO₂ as a distension gas resulted in lower incidents of intra-procedural and postoperative pain. Yet even with CO₂ as the distension gas other undesirable consequences arise, ranging from colon spasm (peristalsis) to mortality risk. Another drawback to the current method is that, as with any insufflation system, a prevalent issue is leaks that can arise in the various seals, thus causing distension to be lost during a procedure. The smoke caused by electro-surgery will cloud the visual space, and must be removed. With the sealed systems currently used, smoke is ported out of the system, and this causes further complication. It has also been observed that the surgeon can easily lose his or her sense of location during a procedure.

Inflatable devices have been used for various medical procedures, with major categories of such procedures being occlusion, dilation, anchoring, and combinations thereof. Several sub-categories also exist in both intra-luminal and extra-luminal uses. Some inflatable devices have been used or at least proposed for purposes of assisting visualization and access within a lumen during a medical procedure. However, these devices nevertheless are in need of improvement.

There is a need for improvements directed to the creation of operative spaces in a patient without the use of distension gas. Such improvements would, for example, improve visualization and access, as well as improve the doctor's ability to use various instruments during a surgical or diagnostic procedure within a body lumen and/or address the drawbacks of current systems, devices and related methods.

### SUMMARY

The present invention pertains to the creation of a viable diagnostic and operative site in a body lumen without the use of a distension gas. In general, the viable operative site is created by positioning a balloon element distal to the site of interest; the balloon thus acting as a distal lumen support A cannula, which is proximal to the site, can serve as a proximal lumen support. For certain instances, such as where the site of interest is high in the colon, a balloon element is placed both proximally and distally to the site, thus giving support to the body lumen on both sides of the site. Minimally invasive surgery, and specifically minimally invasive transanal colorectal surgery, more commonly known by the acronym TEM (transanal endoscopic microsurgery) is especially suited to the use of an inflatable device of this invention for creating an operative space in the GI tract. Regarding TEM procedures, for example, the device eliminates the need for a pressure-tight cannula system and instead gives rise to the use of an open transanal cannula. This method also allows for a smaller cannula than is used in standard practice, thus reducing patient trauma and reduced set-up time.

Generally, the device comprises a balloon element including a one-way valve for inflation air ingress or entry. An air ingress conduit is removably attachable to an ingress port communicating with the one-way air ingress valve. Air egress or exit from the balloon element (e.g., to vent the air after a procedure) may occur through a deliberate puncture to the balloon element. However, the device preferably includes both an ingress and egress port and associated valves. The valves may be constructed with a combined structure that performs both ingress and egress functions

In a particular illustrative embodiment, the invention provides a device for creating an operative space within a body lumen for the performance of a medical procedure. The device comprises a pressurizable, expandable balloon element insertable into the body lumen. The balloon element includes an interior space for receiving an inflation fluid. An egress valve is coupled with the balloon element and is insertable therewith into the body lumen. The ingress valve is capable of being opened to allow the interior space to be filled with the inflation fluid and closed to retain the inflation fluid in the interior space. Preferably, this ingress valve is a one-way valve that automatically opens and closes due to air pressure that is present on inlet and outlet sides of the valve. An egress valve is also coupled with the balloon element in this embodiment and insertable therewith into the body lumen. The egress valve is closed when the interior space is filled with the inflation fluid and is capable of being opened to allow the inflation fluid to vent from the interior space. The device may further comprise a valve body and the ingress and egress valves may be contiguously attached to the valve body thereby forming a unitary valve structure coupled with the balloon element. Preferably, the valve body is formed of a material that is more rigid than the material forming the balloon element. The balloon element may be formed in any desirable shape, however, one advantageous shape is that of a toroid or generally a donut shape. In this regard, the toroid may be a full, continuous toroid or may be a partially toroidal shape having one or more discontinuities in the toroid. The valve body may form a section of the toroid, such as an inner diameter of the toroid.

More specifically, the ingress valve may include an ingress attachment port for attaching an ingress conduit thereto. The egress valve may include an egress valve actuation element for actuating the egress valve. The ingress attachment port and the egress valve actuation element may each be angled approximately 20° with respect to a central axis of the balloon element. In this manner, the ingress attachment port and egress valve actuation or vent element will also be angled approximately 20° to the axis of the body lumen, for easy access to these components. This desirable angle may also be used for the valve engagement component(s) of other embodiments. More broadly stated, the angle is an acute angle that is nonparallel to the central axis of the balloon element and the body lumen. In the case of a toroidal or partially toroidal balloon element, the central axis of the toroid shape is the central axis of the balloon element. In addition, this axis is intended to be generally parallel or coaxial with the axis of the body lumen during use. This preferred angular orientation allows easier access by a medical professional, such as the surgeon, to both the ingress attachment port and the egress valve actuation element during the surgical procedure. The ingress attachment port is preferably internally tapered to allow for a removable press-fit with an externally tapered distal end element of the ingress conduit. The ingress valve includes an opening sized to prevent pressure build-up during inflation of the balloon element that would cause premature detachment of the ingress conduit. The inner diameter portion of the balloon element may include a wall that is thicker than an outer diameter portion of the balloon element. This provides added support for the balloon element and ensures that the inflation or expansion of the balloon element takes place in generally a radially outward direction against the walls of the body lumen for a secure fit and stability during the surgical procedure.

In another illustrative embodiment, a device for creating an operative space within a body lumen includes a balloon element as generally described above in combination with an egress valve. This embodiment may also have an ingress valve, or the egress valve may be a combination ingress/egress valve. The egress valve is coupled with the balloon element and insertable therewith into the body lumen, and includes an egress path that is closed when the interior space of the balloon element is filled with inflation fluid. The egress valve is capable of being opened to allow the inflation fluid to vent from the interior space. The egress valve further includes a vent element forming a pressure type fit in the egress path to retain the inflation fluid in the interior space. The vent element is movable to vent the inflation fluid from the interior space through the egress path. In one embodiment, the vent element further comprises a push-pull element capable of being repeatedly pushed and pulled to open and close the egress path as necessary during the surgical procedure. It will be appreciated that the push-pull element may be pushed to open the egress path or pulled to close the egress path, or vice versa depending on the particular chosen design. In another embodiment, the egress valve further comprises a duck bill valve that includes a vent element. The vent element may be a portion of a duck bill valve, such as a resilient or compliant portion with a spring actuation element that is squeezed to open two duck bill flaps associated with the valve. This duck bill valve preferably serves as a combined ingress/egress valve. In either case, for example, the vent element is capable of being repeatedly actuated to open and close the egress path as necessary during the surgical procedure.

In another illustrative embodiment, a method is provided for creating an operative space within a body lumen and performing a medical procedure in the body lumen. The method includes inserting a first balloon element, including an ingress valve and an egress valve into the body lumen with the balloon element in an unexpanded state. The first balloon element is expanded by introducing an inflation fluid into the first balloon element through the ingress valve to create the operative space. A medical procedure, such as a diagnostic or surgical procedure, is then performed in the operative space. After the procedure is complete, or if necessary, during the procedure, the inflation fluid is vented through the egress valve. Expanding the first balloon element can further comprise inserting an ingress conduit into the body lumen and operatively coupling a distal end of the ingress conduit to the ingress valve, introducing the inflation fluid through the ingress conduit and the ingress valve into the first balloon element, detaching the ingress conduit from the ingress valve, and withdrawing the distal end of the ingress conduit from the operative space. Venting the inflation fluid can further comprise inserting an elongate element into the body lumen and operatively engaging a distal end of the elongate element with a vent element of the egress valve, and moving the vent element by use of the elongate element to vent the inflation fluid from the first balloon element. The first balloon element may then be withdrawn from the body lumen using the elongate element or another instrument. The method can further comprise inserting a second balloon element, including a second ingress valve and second egress valve into the body lumen with the second balloon element in an unexpanded state. The second balloon element is then expanded by introducing an inflation fluid into the second balloon element through the second ingress valve to create the operative space between the first and second balloon elements. The procedure is then performed in the operative space between the first and second balloon elements. After the procedure is complete, the inflation fluid is vented through both the first and second egress valves and the first and second balloon elements are withdrawn from the body lumen.

In another embodiment, a method is provided for creating an operative space within a body lumen and performing a surgical procedure in the body lumen. This method comprises inserting a first balloon element including a combined ingress/egress valve into the body lumen with the first balloon element in an unexpanded state. The first balloon element is then expanded by introducing an inflation fluid into the first balloon element through the combined ingress/egress valve to create the operative space. A medical procedure is then performed in the operative space. After completion of the procedure, or if necessary during the procedure, the inflation fluid is vented through the combined ingress/egress valve.

The combined ingress/egress valve can further include an egress path that is closed when the interior space is filled with the inflation fluid and is capable of being opened to allow the inflation fluid to vent from the interior space. The egress valve further includes a vent element and the method then further comprises inserting an elongate element into the body lumen and moving the vent element with a distal end of the elongate element to vent the inflation fluid from the interior space through the egress path. As mentioned, the vent element may further comprise a push-pull element or a portion of a duck bill valve, for example, or any other suitable structure. The combined ingress/egress valve can further include an egress valve actuation element and in this case the method further comprises operatively engaging the egress valve actuation element with the elongate element while the egress actuation element is at an acute angle of, for example, approximately 20 degrees with respect to a central axis of the body lumen.

The embodiments of this invention further provide for a system whereby devices of the present invention are combined with one or more device introduction cannula(s), ingress conduit(s), elongate element(s), such as grasper tools or other instruments, for actuating the egress valve structure, and a pump or other air introduction device for inflating the balloon element(s).

Additional aspects, features and advantages will become more readily apparent to those of ordinary skill upon review of a detailed description of illustrative embodiments of the invention below, taken in conjunction with the accompanying drawings.

### BRIEF DESCRIPTION OF THE DRAWINGS

FIG. 1 is a perspective view of a first embodiment of a system including a device of the present invention incorporated into a system shown with an ingress conduit and an air pump, and showing the device inserted into a body lumen, such as the colon, and the pump located outside the body lumen.

FIG. 2 is an enlarged perspective view of the device shown in FIG. 1, including a press-fit coupling element for the air ingress conduit.

FIG. 3 is a cross section view taken along line 3-3 of FIG. 2 and showing the balloon element in dash-dot lines.

FIG. 4A is a cross sectional view taken along line 4A-4A of FIG. 3 and showing the egress valve in a closed or non-venting position.

FIG. 4B is a cross sectional view similar to FIG. 4A, but illustrating the egress valve in an open or venting position.

FIG. 5 is a perspective view of another embodiment of a device constructed in accordance with a second embodiment of the invention.

FIG. 6A is a cross sectional view taken along line 6A-6A of FIG. 5 showing air being introduced into the balloon element through the ingress valve, and the egress valve closed.

FIG. 6B is a cross sectional view similar to FIG. 6A, but illustrating the egress valve opened and venting the air from the balloon element.

FIG. 7 is a perspective view of a device constructed in accordance with another embodiment of the invention.

FIG. 8A is a cross sectional view taken along line 8A-8A of FIG. 7 and illustrating pressurized air being introduced into the balloon element through a duck bill ingress/egress valve.

FIG. 8B is a cross sectional view similar to FIG. 8A, but illustrating a grasper tool or instrument being used to open the duck bill ingress/egress valve to vent the air from the balloon element.

FIG. 9 is a perspective view of the spring actuation element used to open and close the duck bill valve.

FIG. 10 is a perspective view of a device constructed in accordance with another illustrative embodiment of the invention.

### DETAILED DESCRIPTION OF THE ILLUSTRATIVE EMBODIMENTS

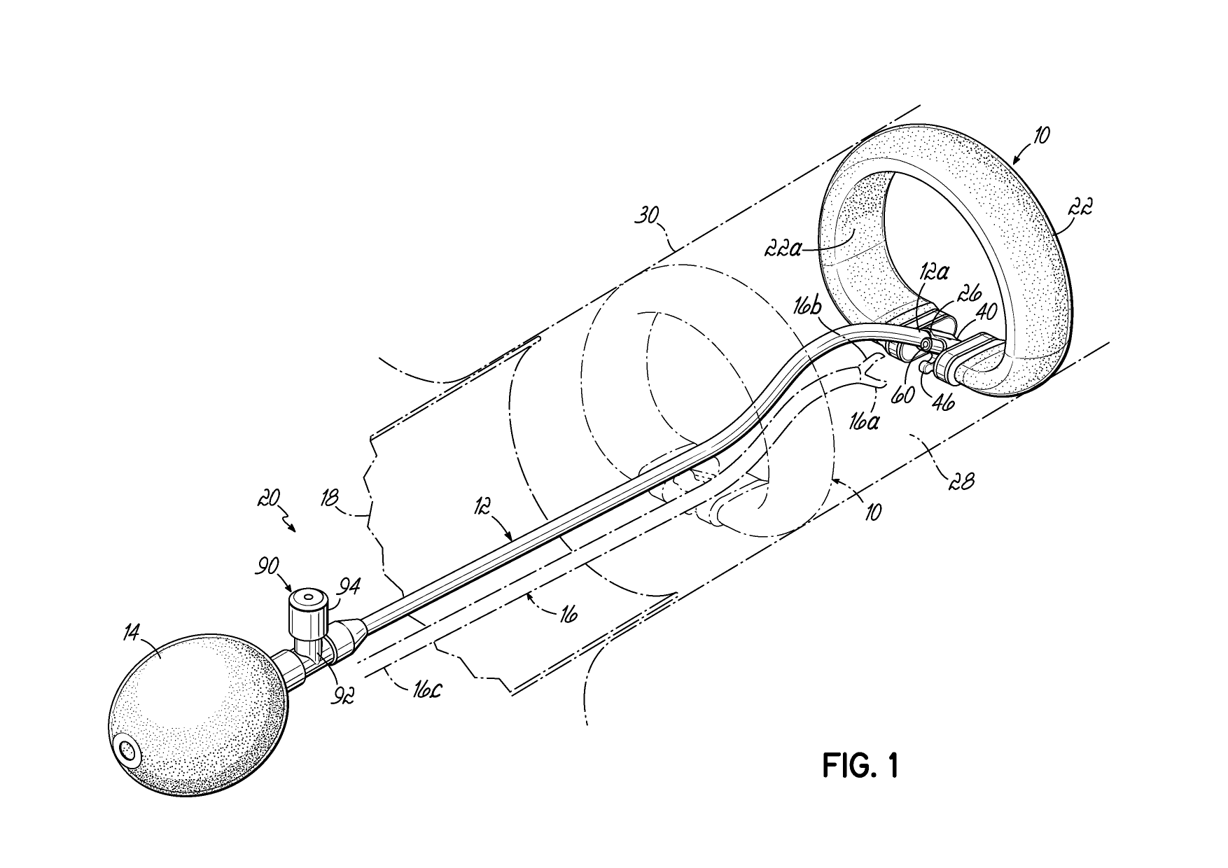

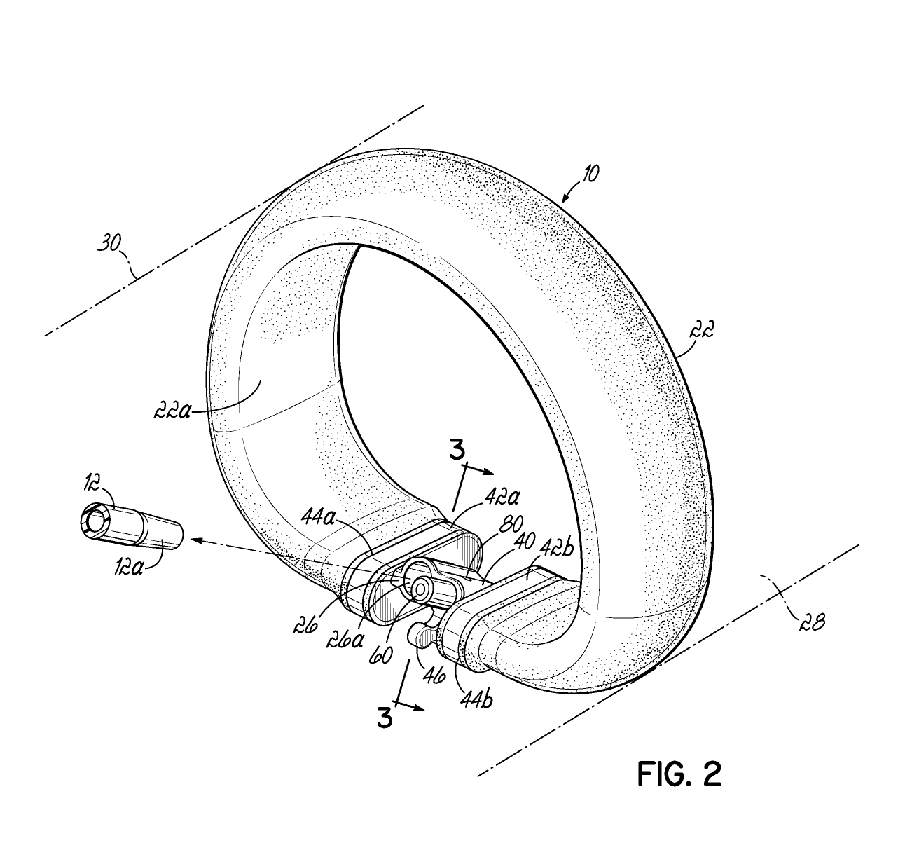

Referring to FIGS. 1 and 2, a device 10 is shown and constructed in accordance with a first embodiment of the invention. FIG. 1 illustrates the device 10 coupled with an ingress conduit 12, a pump 14, an elongate grasper tool 16, and an introduction cannula 18 for completing one embodiment of a system 20 constructed in accordance with the invention. The pump 14 and ingress conduit 12 are used to introduce expansion or inflation fluid, such as air, into a balloon element 22 of the device 10. The pump 14 may take any desired form, but in this embodiment the pump 14 is shown as a squeeze bulb. Any suitable air introduction device may be used. A distal end 12a of the ingress conduit 12 is coupled to an ingress port 26, best illustrated in FIG. 2. For example, the distal end 12a of the ingress conduit 12 may include an outer taper that is press-fit to a tapered inner surface 26a of the port 26 to allow temporary attachment of the ingress conduit 12 and squeeze bulb pump or other air introduction device 14 to the device 10 for inflation purposes. Once adequate inflation is achieved, the ingress conduit 12 is detached from the ingress port 26 leaving the operative space 28 proximal to the device 10 unobstructed for diagnostic and/or surgical procedures. In certain cases a second, more proximally located device 10 (shown in dashed lines) may be used with the operative space 28 thereby located between two devices 10.

The balloon element 22 may comprise an elastomeric or other compliant material, or may comprise a non-compliant but still flexible material. Compliant or elastomeric materials may, for example, include thermoplastic elastomers such as polyurethane, polyethylene or latex, while non-compliant materials may include Nylon, PET or like materials that still have adequate flexibility for the purposes described herein. It will appreciated that any other suitable, bio-compatible materials may be chosen instead. The balloon element 22, in this embodiment, forms a generally annular or toroidal shape. As illustrated in FIGS. 1 and 2, the balloon element 22 itself is partially toroidal, due to the interruption in the toroid caused by the valve structure, to be described in detail below. Compliance or flexibility of the balloon element 22 will allow for size variations of the body lumen 30 into which the balloon element 22 is introduced and expanded. The thickness of the wall of the balloon element 22 together with the choice of material will give appropriate burst strength for the desired application. The balloon element 22 has a thicker wall section 22a on the inner diameter to create a stable inner diameter and ensure radial outward expansion against the wall of the lumen 30, such as the colon wall. The added thickness will also provide greater protection against inadvertent puncture.

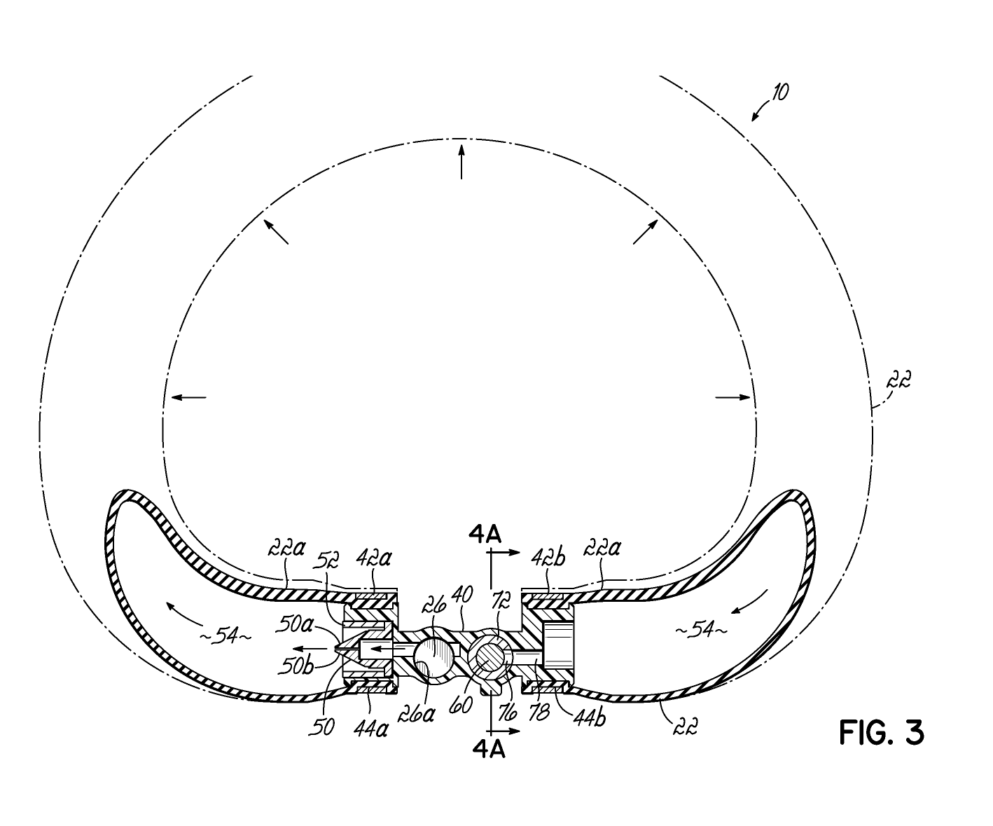

As shown best in FIGS. 2 and 3, the balloon element 22 is affixed in a pressure tight manner to a valve body 40 using elastic elements or mechanical bands, or other structure. The elastic elements 42a, 42b or mechanical bands fit within respective grooves 44a, 44b on the valve body 40. A knob or projection 46 is provided on the valve body 40 to enable grasping, manipulation and/or other handling of the device 10 using the grasper 16. Although such structure may not be shown on other embodiments disclosed herein, it will be understood that it may be provided on any embodiment of the invention. The outer surface of the balloon element has a surface texture and shape allowing it to optimally anchor within the lumen 30. The valve body 40 is comprised of bio-compatible polymer material, such as medical grade Delrin. The other components of the valve structure are also formed of any suitable medical grade materials.

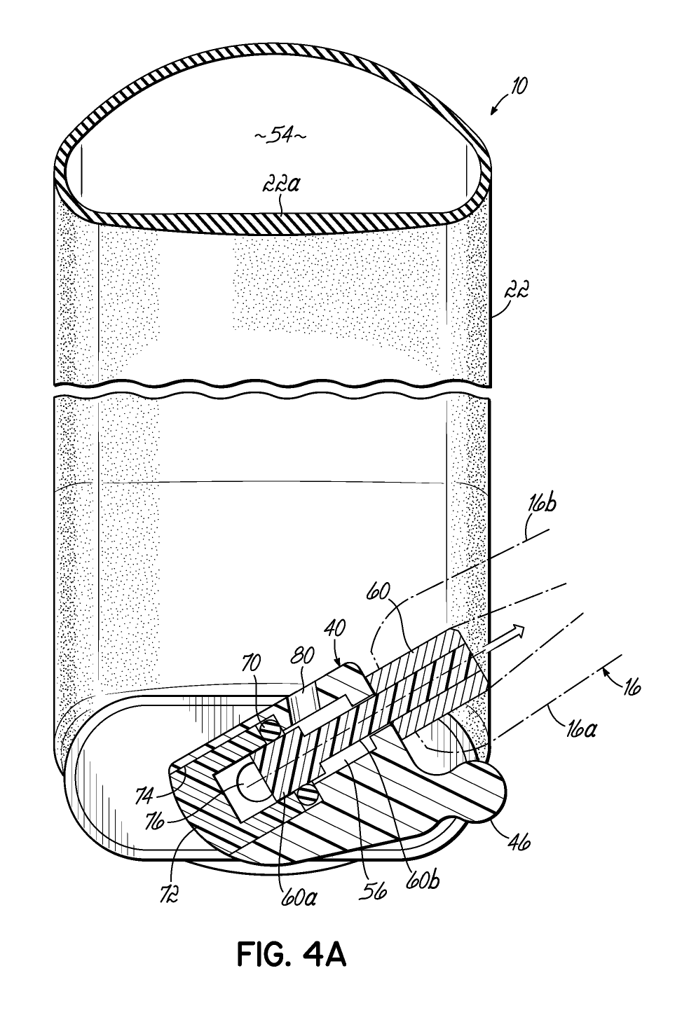

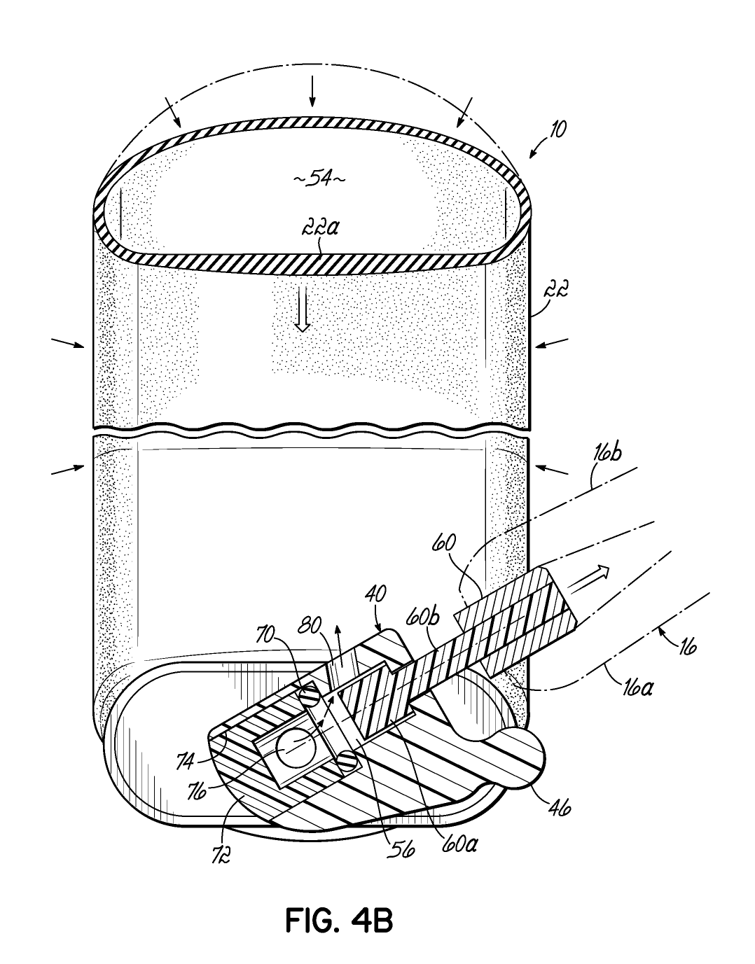

Now referring to FIGS. 3, 4A and 4B, the ingress valve 50 is shown in FIG. 3 and is secured in the valve body 40 by a valve retainer 52. A pressure-tight seal is created by pressing the valve retainer 52 against the valve body and securing it in place by means of ultrasonic weld, epoxy or any other suitable method. The ingress air passes from the pump 14 and conduit 12 into the ingress port 26 and flows into the interior space 54 of balloon element 22. When the air stops flowing through the ingress valve 50, two opposed duck bill portions or flaps 50a, 50b of the valve 50 will automatically close thereby sealing the balloon element 22 in an inflated state. As shown in FIGS. 4A and 4B, the valve body 40 includes a chamber 56 that receives an egress actuation or vent element 60 and controls venting of air from the interior space 54 of the balloon element 22. The vent element 60 is a push-pull element in this embodiment, however, it will be appreciated that other types of movable valve elements may be used instead. In the open position, shown in FIG. 4B, the air will flow from the interior space 54 to the environment, i.e., into the body lumen 30. Venting of the air is stopped when the base 60a of the vent element 60 is seated in a sealing manner within an O-ring 70. The O-ring 70 is held in place by a cap 72 that is housed in the valve body 40 and seated in a counterbore 74. The cap 72 includes a hole 76 on its side that aligns with a port 78 (FIG. 3). As the valve stem 60b is retracted by the elongate grasping instrument or tool 16 inserted into the body lumen 30, the vent element 60 moves fully into the chamber 56 thereby dislodging the base 60a from the O-ring 70. This allows for a continuous egress path through the port 78, hole 76, egress chamber 56 and, finally, an exhaust port 80. The base 60a moves to a proximal stop position within the egress chamber 56 and continued pulling of the grasper tool 16 will cause the device 10 to be withdrawn from the patient.

Referring again to FIG. 1, the pump 14, as mentioned, may consist of a squeeze bulb or other suitable fluid pump. If the squeeze bulb 14 is sized to require multiple pumping cycles to fill the balloon element 22, then it must be fitted with a check valve (not shown) that allows the pump 14 to force fluid, such as air, through the ingress conduit 12 and then allows the squeeze bulb 14 to be refilled from an external source, such as room air. The pump 14 will have an overpressure device 90, such as a check valve, to gauge the amount of pressure the balloon element 22 is placing on the anatomy. When a preselected pressure is reached, the overpressure valve 90 will release indicating the balloon element 22 is adequately pressurized. One embodiment of a check valve includes a spring (not shown) used to elastically hold a ball (not shown) against an opening in a pump stem 92. A cap 94 is threaded onto the pump stem 92 to vary the amount of spring force on the ball and thus acts to meter the amount of pressure that is delivered to the balloon element 22 through the ingress valve 50.

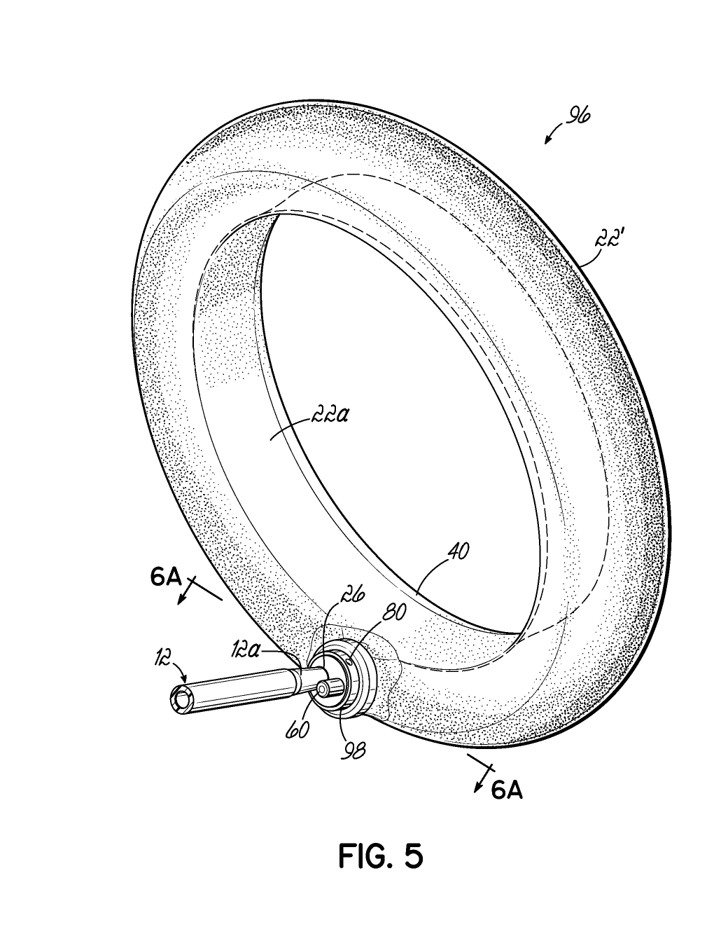

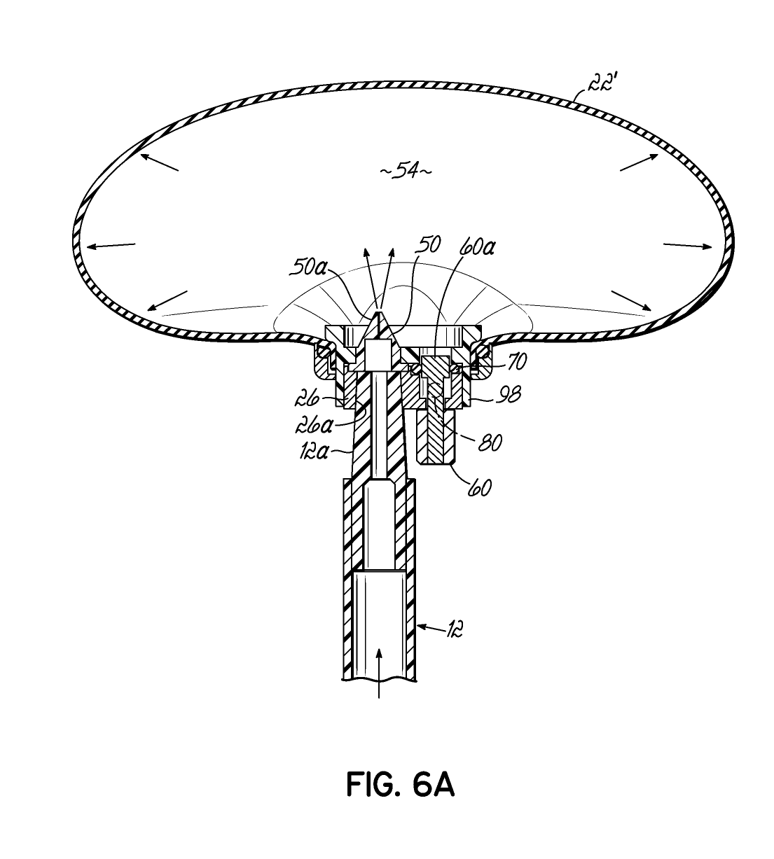

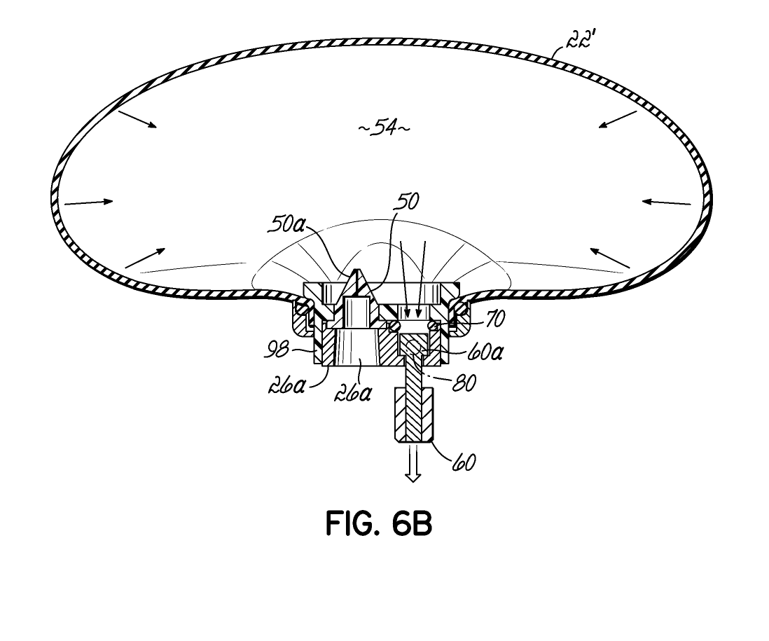

FIGS. 5, 6A and 6B illustrate another embodiment of a device 96 constructed in accordance with the invention. This embodiment includes valve structure that is very similar to the valve structure shown best in FIGS. 4A and 4B. Therefore, like elements or components of the embodiments, having like function, have been denoted in these figures with like reference numerals. Additional detailed description therefore is not necessary. This embodiment illustrates that the toroidal balloon element 22′ may be essentially continuous, instead of discontinuous as shown in the first embodiment, and that the valve structure 98 may be essentially contained within and attached to a sidewall of the balloon element 22′.

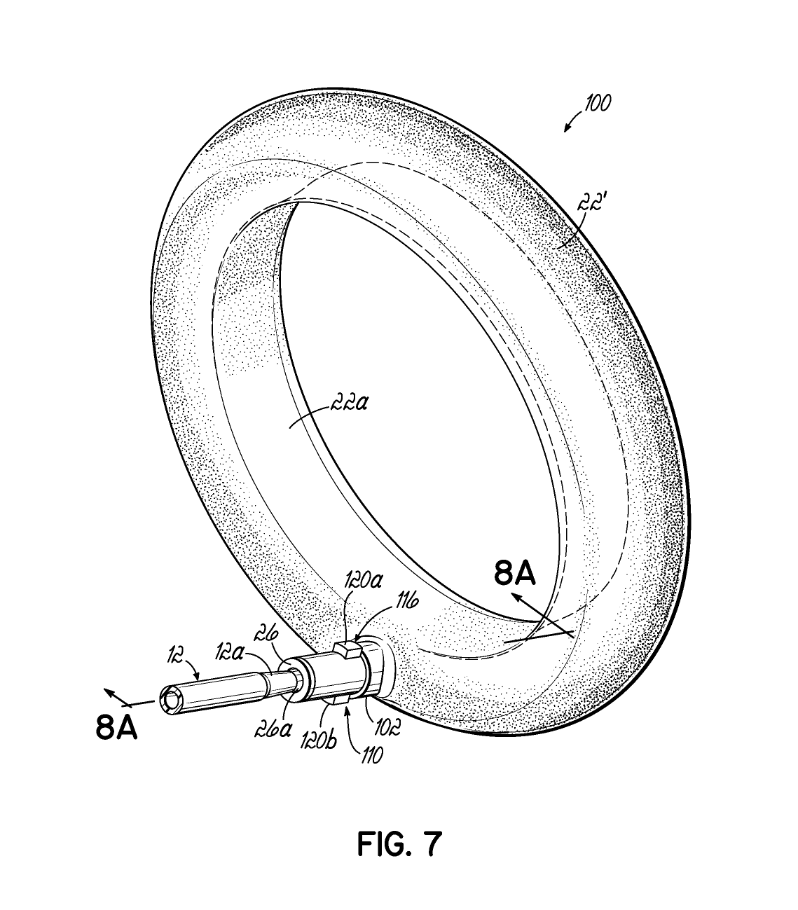

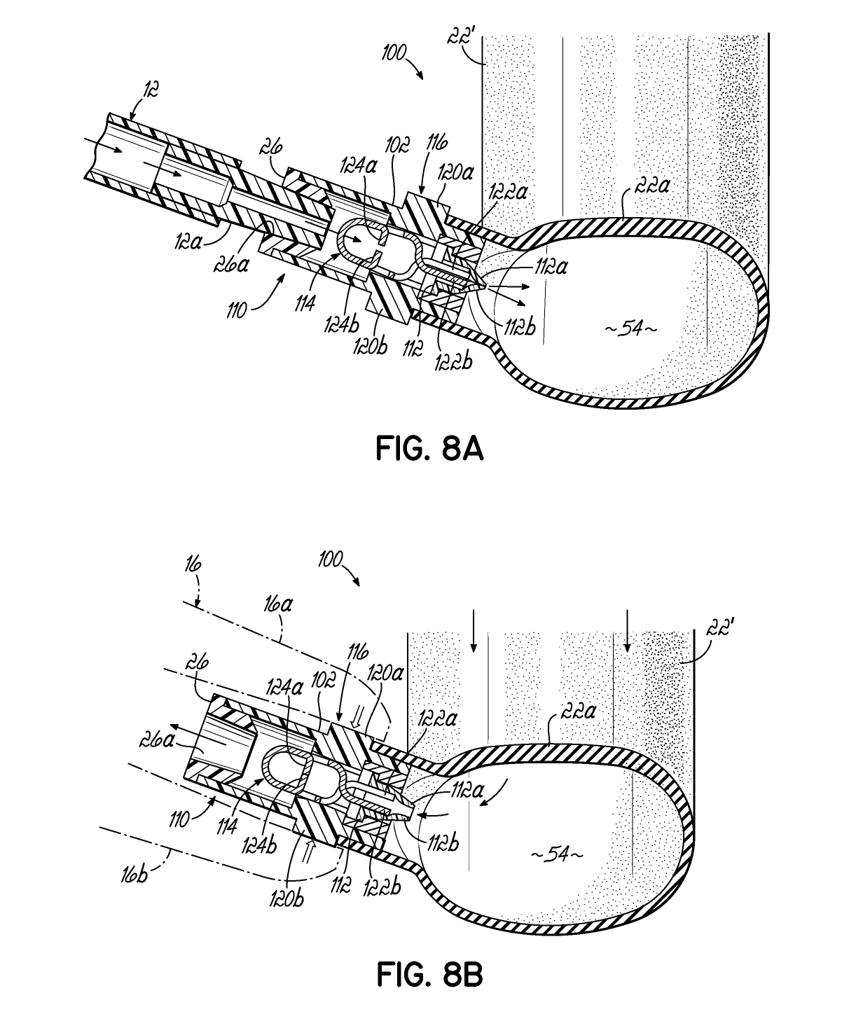

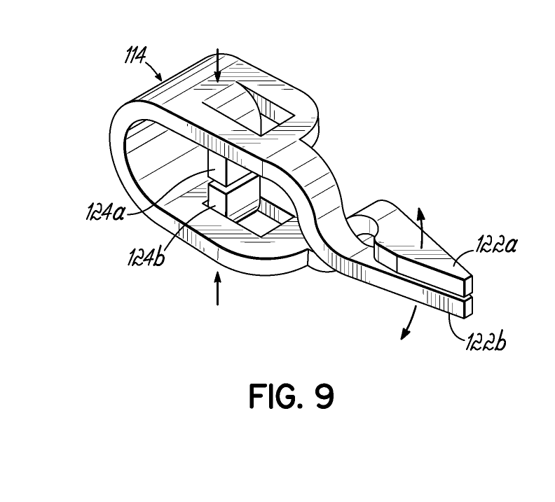

FIGS. 7, 8A, 8B and 9 illustrate a device 100 constructed in accordance with another embodiment. This device is similar to the device 96 shown in FIG. 5 in that the balloon element 22′ is essentially continuous with a valve body 102 attached to and extending from a sidewall of the balloon element 22′. In this embodiment, however, the ingress and egress valves are incorporated into a combined ingress/egress valve 110 shown best in FIGS. 8A and 8B. In this regard, the combined ingress/egress valve 110 is a duck bill valve having a duck bill valve element 112 actuated by a spring actuation element 114 (see FIG. 9 also). As shown in FIG. 8A, pressurized air is introduced through the ingress conduit 12 and distal end attachment fitting 12a with a tapered press-fit that detachably couples the ingress conduit 12 to the ingress port 26. Air is directed past the actuation element 114 and through the duck bill valve element 112. As with the previously described duck bill valve element 50, respective portions or flaps 112a, 112b of the duck bill valve element 112 spread apart and open under the influence of the pressurized air to allow the air into the interior space 54 of the balloon element 22. When the air is no longer being introduced, the duck bill valve portions or flaps 112a, 112b close and seal against each other to prevent the air within the interior space 54 from escaping. When it is desired to vent the air from the interior space 54, a suitable elongate grasper tool 16 is inserted into the body lumen 30 and the grasper members 16a, 16b, as shown in FIG. 8B, squeeze together against a compliant outer portion 116 of the valve body 102 and specifically against a pair of pads 120a, 120b on the outer portion 116 of the valve body 102. This action actuates the spring actuation element 114 of the valve 110 to cause a pair of distal fingers 122a, 122b to spread apart and engage the respective duck bill valve portions or flaps 112a, 112b. This spreads the duck bill valve portions or flaps 112a, 112b apart as shown in FIG. 8B and allows the air within the interior space to vent, thereby deflating the balloon element. Stops 124a, 124b are also provided to positively stop the spring actuation element 114 at a predetermined point. It should be appreciated that the spring actuation element 114 may take other forms. This includes structure molded into or otherwise formed as a more integral part of the valve 110.

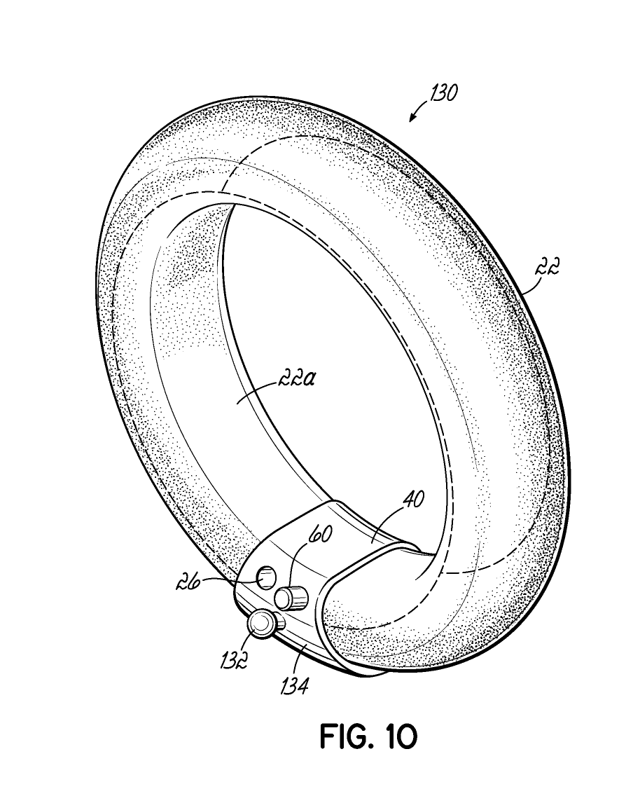

FIG. 10 illustrates yet another embodiment of a device 130. This embodiment includes a similar balloon and valve structure to the structures shown and described with regard to the first two embodiments. And, in those regards, further description is not necessary. This embodiment further illustrates a projection 132 that may be used by a medical professional, such as a surgeon, to grasp, orient, and test the holding power of the device 130 in the body lumen 30. In addition, a valve body casing 134 is provided in generally an annular shape surrounding, or partially surrounding, a portion of the toroidal balloon element 22 to generally form the device 130 into a complete toroid while providing additional support for interior valve components.

A device of the invention, such as device 10, is prepared and introduced to the target site in the following steps. The device 10 is placed in the grasper jaws 16a, 16b and is grasped by engaging and grasping the valve body 40. The area on the valve body 40 that is grasped can have features that facilitate grasping with grasper 16, such as knob 46 (FIG. 2). These features may include a small ridge that the grasper teeth can bite into for solid purchase. The ingress conduit 12 is aligned along the grasper shaft 16c. The ingress tube or conduit 12 may be wrapped around or otherwise attached to the grasper shaft 16c during the insertion procedure. The tapered distal end or fitting 12a is then fitted into the ingress port 26. The internal passage of the fitting 12a is sized to prevent over-pressure between the fitting 12a and the valve 50, which could cause the fitting 12a to detach prematurely. The device 10 is now ready for introduction.

The introduction cannula 18 is first placed into the body lumen 30, such as a lumen of the GI tract, and the device 10 is introduced through the cannula 18 into the lumen 30. An endoscopic camera and grasper, optical forceps or similar instruments (not shown) may be used to help accurately place the device 10 at the target site. Once the site has been reached, the pump 14 is actuated to inflate the balloon element 22 until the desired pressure condition is attained or the over-pressure valve or device 90 is activated.

Once the balloon element 22 is inflated, the grasper 16 can be used to pull on the device 10 to check the integrity of the hold. The grasper 16 is then released from the valve body 40. The grasper 16 then removes the fitting 12a from the valve body 40. The grasper 16 and associated ingress conduit 12 are removed from the body through the cannula 18, and the desired medical procedure(s) is/are performed using instruments directed through the cannula 18. Advantageously, the conduit 12 and pump 14 are operated from outside the body of the patient, as is the grasper 16. Also, since the conduit 12 and grasper 16 need not be present during the actual medical procedure(s), they will not present obstacles during the procedure(s).

Once the procedure(s) is/are complete and the device 10 is ready for removal, the grasper 16, without the ingress conduit 12, is reintroduced into the lumen 30. The grasper 16 is used to grasp the vent element 60 and the doctor pulls proximally. This action causes the vent element 60 to move proximally into the valve body egress chamber 56. When the vent element 60 passes the egress port 78 the inflation fluid (e.g., air) in the interior space 54 escapes and the balloon element 22 deflates. At this point the doctor can pull on the grasper 16 until the device 10 exits the cannula 18, or the doctor can re-grasp the valve body for removal of the device 10.

Various modifications or substitutions may be made to the details described herein. As non-limiting examples, the pump can have various styles and configurations including: a syringe that connects to the ingress tube by a suitable fitting; a bulb that connects by a releasable fitting (making the pump reusable); a syringe with valves that enable cycling without the plunger of the syringe being removed; an umbrella valve for a pre-set overpressure of the inflation gas; a gauge that reads the current pressure of the gas; and a rotary style pump. Polymer components may be ultrasonically welded or epoxied in hermetic fashion. The vent element can be a rotary type element instead of push-pull.

This invention has a number of distinct advantages to current operative procedures. For example, an open cannula system offers much more aggressive smoke evacuation during electro-surgery. With a pressurized system the smoke created by electro-surgery will cloud the field of view. An open cannula offers the opportunity for greater evacuation of smoke. Also, to pass a needle into the body lumen in a pressurized system, the sealing cap must be removed, the needle passed, and the seal re-seated. With this device an opening is already available. The occurrence of peristalsis or colon spasm, which impairs visualization, is also reduced or eliminated. Seals on a pressurized system will often leak due to inadequate seating or due to a defect. Devices of the present invention eliminate this issue. Further, the removal of excised material is not an issue with devices and methods of this invention. Surgical and diagnostic instruments are not constrained by devices of this invention. This allows for true tactile feedback as well as unrestricted motion of the instruments. Thus, the instruments can approach the site from different angles in an unrestricted manner. In addition, a smaller rectoscope cannula may be used with a device of this invention. Finally, any time insufflation gas is removed from a procedure, as with the present inventive devices and methods, associated possibility of embolisms is removed.

While the present invention has been illustrated by a description of various preferred embodiments and while these embodiments have been described in some detail, it is not the intention of the Applicants to restrict or in any way limit the scope of the appended claims to such detail. Additional advantages and modifications will readily appear to those skilled in the art. The various features of the invention and the various embodiments may be used alone or in any combination depending on the needs and preferences of the user. This has been a description of the present invention, along with the preferred manners of practicing the present invention as currently known. However, the invention itself should only be defined by the appended claims.

What is claimed is:

1. A device for creating an operative space within a body lumen for the performance of a medical procedure, comprising:
a pressurizable, expandable balloon element insertable into the body lumen, said balloon element including an interior space for receiving an inflation fluid,
an ingress valve coupled with said balloon element and insertable therewith into the body lumen, wherein said ingress valve is capable of being opened to allow the interior space to be filled with the inflation fluid and closed to retain the inflation fluid in the interior space, and
an egress valve coupled with said balloon element and insertable therewith into the body lumen, wherein said egress valve is closed when the interior space is filled with the inflation fluid and is capable of being opened to allow the inflation fluid to vent from the interior space.

2. The device of claim 1, further comprising:
a valve body, wherein said ingress and egress valves are contiguously attached to said valve body.

3. The device of claim 1, wherein said balloon element is at least partially in the shape of a toroid.

4. The device of claim 3, further comprising:
a valve body, wherein said ingress and egress valves are contiguously attached to said valve body, and said valve body forms a section of the toroid.

5. The device of claim 4, wherein said valve body defines an inner diameter of the toroid.

6. The device of claim 1, wherein said ingress valve includes an ingress attachment port for attaching an ingress conduit thereto, and said egress valve includes an egress valve actuation element for actuating said egress valve, wherein said ingress attachment port and said egress valve actuation element are each oriented at an acute angle with respect to a central axis of the balloon element.

7. The device of claim 1, wherein said ingress valve includes an ingress attachment port for attaching an ingress conduit thereto, said ingress attachment port being tapered to allow for a removable press-fit with an ingress conduit.

8. The device of claim 1, wherein said ingress valve includes an opening sized to prevent pressure build-up during inflation that would cause premature detachment of an ingress conduit.

9. The device of claim 1, wherein said balloon element is at least partially in the shape of a toroid, and an inner diameter portion of said balloon element includes a wall that is thicker than an outer diameter portion.

10. A device for creating an operative space within a body lumen for the performance of a medical procedure, comprising:
a pressurizable, expandable balloon element insertable into the body lumen, said balloon element including an interior space capable of receiving an inflation fluid, and
an egress valve coupled with said balloon element and insertable therewith into the body lumen, wherein said egress valve includes an egress path that is closed when the interior space is filled with the inflation fluid and is capable of being opened to allow the inflation fluid to vent from the interior space, said egress valve including a vent element, wherein said vent element is movable to vent the inflation fluid from the interior space through the egress path.

11. The device of claim 10, wherein said vent element further comprises a push-pull element, wherein said push-pull element is capable of being repeatedly pushed and pulled to open and close said egress path as necessary during the medical procedure.

12. The device of claim 10, wherein said egress valve further comprises a duck bill valve and said vent element further comprises a portion of said duck bill valve, wherein said portion of the duck bill valve is capable of being squeezed to open said egress path as necessary during the medical procedure.

13. The device of claim 10, wherein said egress valve includes an egress valve actuation element for actuating said egress valve, wherein said egress valve actuation element is oriented at an acute angle with respect to a central axis of the balloon element.

14. A device for creating an operative space within a lumen for the performance of a medical procedure, comprising:
a pressurizable, expandable balloon element insertable into the body lumen, said balloon element including an interior space capable of receiving an inflation fluid, and
an ingress valve coupled with said balloon element and insertable therewith into the body lumen, wherein said ingress valve includes an ingress attachment port comprising structure allowing for the removable attachment of an ingress conduit thereto, whereby the ingress conduit may be introduced into the body lumen and attached to said attachment port to inflate said balloon element and then detached from said attachment port while retaining said balloon element in an inflated state, thereby preventing obstruction of an operative site by the ingress conduit during the medical procedure.

15. A method for creating an operative space within a body lumen and performing a medical procedure in the body lumen, comprising:
inserting a first balloon element, including an ingress valve and an egress valve into the body lumen with the first balloon element in an unexpanded state,
expanding the first balloon element by introducing an inflation fluid into the first balloon element through the ingress valve to create the operative space,
performing the medical procedure in the operative space, and venting the inflation fluid from first balloon element through the egress valve.

16. The method of claim 15, wherein expanding the first balloon element further comprises:
inserting an ingress conduit into the body lumen and operatively coupling a distal end of the ingress conduit to the ingress valve, introducing the inflation fluid through the ingress conduit and the
ingress valve into the first balloon element,
detaching the ingress conduit from the ingress valve, and
withdrawing the distal end of the ingress conduit from the operative space.

17. The method of claim 15, wherein venting the inflation fluid further comprises:
inserting an elongate element into the body lumen and operatively engaging a distal end of the elongate element with a vent element of the egress valve, and
moving the vent element by use of the elongate element to vent the inflation fluid from the first balloon element.

18. The method of claim 17, further comprising:
withdrawing the first balloon element from the body lumen using the elongate element.

19. The method of claim 15, further comprising:
inserting a second balloon element, including a second ingress valve and a second egress valve into the body lumen with the second balloon element in an unexpanded state,
expanding the second balloon element by introducing an inflation fluid into the second balloon element through the second ingress valve to create the operative space between the first and second balloon elements,
performing the medical procedure in the operative space between the first and second balloon elements,
venting the inflation fluid through both the first and second egress valves, and
withdrawing the first and second balloon elements from the body lumen.

20. A method for creating an operative space within a body lumen and performing a medical procedure in the body lumen, comprising:
inserting a first balloon element including a combined ingress/egress valve into the body lumen with the first balloon element in an unexpanded state,
expanding the first balloon element by introducing an inflation fluid into the first balloon element through the combined ingress/egress valve to create the operative space,
performing the medical procedure in the operative space, and
venting the inflation fluid through the combined ingress/egress valve.

21. The method of claim 20, wherein the combined ingress/egress valve further includes an egress path that is closed when the interior space is filled with the inflation fluid and is capable of being opened to allow the inflation fluid to vent from the interior space, the egress valve including a vent element, and the method further comprises:
inserting an elongate element into the body lumen, and
moving the vent element with a distal end of the elongate element to vent the inflation fluid from the interior space through the egress path.

22. The method of claim 21, wherein the vent element further comprises a push-pull element, and the method further comprises:
pushing or pulling the push-pull element to open the egress path.

23. The method of claim 21, wherein the vent element further comprises a portion of a duck bill valve, and the method further comprises:
actuating the portion of the duck bill valve to open the egress path.

24. The method of claim 21, wherein the combined ingress/egress valve includes an egress valve actuation element, and the method further comprises:
operatively engaging the egress valve actuation element with an elongate element while the egress actuation element is at an acute angle with respect to a central axis of the body lumen.

25. A method for creating an operative space within a body lumen and performing a medical procedure in the body lumen, comprising:
inserting a first balloon element including an ingress valve into the body lumen with the first balloon element in an unexpanded state,
expanding the first balloon element by introducing an inflation fluid into the first balloon element through the ingress valve to create the operative space,
performing the medical procedure in the operative space,
venting the inflation fluid from the first balloon element, and
withdrawing the first balloon element from the body lumen.

26. The method of claim 25, wherein the first balloon element further includes an egress path that is closed when the first balloon element is filled with the inflation fluid and is capable of being opened to allow the inflation fluid to vent from the first balloon element, the first balloon element further including a vent element, and the method further comprises:
inserting an elongate element into the body lumen, and
moving the vent element with a distal end of the elongate element to vent the inflation fluid from the first balloon element through the egress path.

27. The method of claim 26, wherein the vent element further comprises a push-pull element, and the method further comprises:
pushing or pulling the push-pull element to open the egress path.

28. The method of claim 26, wherein the vent element further comprises a portion of a duck bill valve, and the method further comprises:
actuating the portion of the duck bill valve to open the egress path.

29. A system for creating an operative space within a body lumen for the performance of a medical procedure, comprising:
a pressurizable, expandable balloon element insertable into the body lumen, said balloon element including an interior space for receiving an inflation fluid,
an ingress valve coupled with said balloon element and insertable therewith into the body lumen, wherein said ingress valve is capable of being opened to allow the interior space to be filled with the inflation fluid and closed to retain the inflation fluid in the interior space,
an egress valve coupled with said balloon element and insertable therewith into the body lumen, wherein said egress valve is closed when the interior space is filled with the inflation fluid and is capable of being opened to allow the inflation fluid to vent from the interior space,
a cannula configured to receive the balloon element in an unexpanded state for delivery to the body lumen,
an ingress conduit for delivering the inflation fluid to the interior space of the balloon element, and
an elongate element for opening the egress valve.

30. The system of claim 29, wherein said ingress conduit is configured to be releasably coupled to said ingress valve such that said ingress conduit may be removed during the medical procedure.

31. The system of claim 29, wherein said elongate element is configured to be directed into the body lumen after the medical procedure is performed and operatively coupled to the egress valve.

32. A system for creating an operative space within a body lumen for the performance of a medical procedure, comprising:
a pressurizable, expandable balloon element insertable into the body lumen, said balloon element including an interior space capable of receiving an inflation fluid,
an egress valve coupled with said balloon element and insertable therewith into the body lumen, wherein said egress valve including an egress path that is closed when the interior space is filled with the inflation fluid and is capable of being opened to allow the inflation fluid to vent from the interior space, said egress valve including a vent element, wherein said vent element is movable to vent the inflation fluid from the interior space through the egress path,
a cannula configured to receive the balloon element in an unexpanded state for delivery to the body lumen, and
an elongate element for opening the egress valve.

33. The system of claim 32, wherein said elongate element is configured to be directed into the body lumen after the medical procedure is performed and operatively coupled to the egress valve.

34. A system for creating an operative space within a body lumen for the performance of a medical procedure, comprising:
a pressurizable, expandable balloon element insertable into the body lumen, said balloon element including an interior space capable of receiving an inflation fluid,
an ingress valve coupled with said balloon element and insertable therewith into the body lumen, wherein said ingress valve includes an ingress attachment port comprising structure allowing for the removable attachment of an ingress conduit thereto, whereby the ingress conduit may be introduced into the body lumen and attached to said attachment port to inflate said balloon element and then detached from said attachment port while retaining said balloon element in an inflated state, thereby preventing obstruction of an operative site by the ingress conduit during the medical procedure,
a cannula configured to receive the balloon element in an unexpanded state for delivery to the body lumen, and
an ingress conduit for delivering the inflation fluid to the interior space of the balloon element.

35. The system of claim 34, wherein said ingress conduit is configured to be releasably coupled to said ingress valve such that said ingress conduit may be removed during the medical procedure.
